(19) United States
(12) Patent Application Publication
(10) Pub. No.: US 2025/0134426 A1
(43) Pub. Date: May 1, 2025

(54) SUBCUTANEOUS FUNCTIONAL NEAR-INFRARED SPECTROSCOPY

(71) Applicant: Blackrock Microsystems, Inc., Salt Lake City, UT (US)

(72) Inventors: Sherman Perry WIEBE, Brussels (BE); Gillian Eden KOEHL, London (GB); Sandeep NEGI, Salt Lake City, UT (US); Robert Kyle FRANKLIN, IV, Farmington, UT (US); Moritz Michael LEBER, Salt Lake City, UT (US)

(21) Appl. No.: 18/924,904

(22) Filed: Oct. 23, 2024

Related U.S. Application Data
(60) Provisional application No. 63/593,916, filed on Oct. 27, 2023.

(51) Int. Cl. A61B 5/1455; A61B 5/0205; A61B 5/1459; A61B 5/293

(52) U.S. Cl. CPC A61B 5/14553; A61B 5/0205; A61B 5/14552; A61B 5/1459; A61B 5/293; A61B 2562/12

(57) ABSTRACT

Apparatuses, systems, and methods are disclosed for subcutaneous functional near-infrared spectroscopy. An example apparatus includes a substrate and a device configured to be disposed under skin. The device includes emitters and light detectors disposed on the substrate. The emitters emit light that propagates a body part under skin and the light detectors detect changes in light absorption and scattering through the body part and provide fNIRS signals.

### RELATED APPLICATIONS

This application claims priority benefit of U.S. Provisional Patent Application No. 63/593,916, filed Oct. 27, 2023, titled SUBCUTANEOUS FUNCTIONAL NEAR-INFRARED SPECTROSCOPY, which is incorporated herein by reference in its entirety.

### BACKGROUND

Functional near-infrared spectroscopy (fNIRS) is an optical brain monitoring technique using near-infrared light propagating a skull and brain and will be absorbed by chromophores such as oxyhemoglobin and deoxyhemoglobin. Similarly to functional magnetic resonance imaging (fMRI), the fNIRS detects changes in hemoglobin inside the brain. Typically, the fNIRS uses non-invasive optical sources and detectors attached to a body part, such as a head. The detectors detect changes in light absorption and scattering in tissue caused by changes in concentration of oxyhemoglobin and deoxyhemoglobin, which is a consequence of neuronal metabolic activity. The fNIRS has been used in many areas of research and clinical applications for its portability and potential for long-term monitoring, such as a biomarker of major depressive disorders and monitoring of dementia.

Transcutaneous fNIRS measurements have had a problem of near-infrared light absorption and scattering by hair and skin pigment of a head. Additional sources of physiological noise present in the fNIRS measurements include, for example, a heart rate, blood pressure fluctuations, a respiratory rate, and scalp blood flows. The transcutaneous fNIRS measurements suffer from several obstacles. A signal-to-noise ratio (SNR) in fNIRS is often low due to poor optical contact between optical fiber sensors (e.g., optodes) that deliver and collect near-infrared light to and from the scalp. For example, hair of a subject, especially with dark hair colors and high hair root densities, are known to cause poor optical contact. Other environmental factors including presence of ambient light may result in a lower SNR. During setup of optical fiber sensors onto a head, hair on the head may be parted in order for optical fiber sensors to have good optical contact with the scalp. This process is time consuming and becomes more difficult for higher spatial resolution studies with denser probe geometries. Due to these obstacles, many studies have restricted the fNIRS measurements to the prefrontal cortex or to subjects with low hair density. Furthermore, reproducibility in spatial localization of an fNIRS signal source is often limited by a lack of co-registration between the anatomy of a subject and sensor placement on the skin of the subject. Motion artifacts may occur while talking or by movements of a face, a head, and an upper body of a subject. These artifacts may be due to momentary displacement of optical fiber sensors on the scalp, resulting in sharp high frequency displacements, slow wave drifts, or baseline shifts in an fNIRS signal. Various methods have been used to remove motion artifacts, but the results have not been entirely satisfactory. The skin accounts for approximately 50% of the light absorption and scattering in fNIRS measurements, because a near-infrared light absorption coefficient of the skin is much greater than that of the skull. The scalp has a consistently greater influence on brain sensitivity of fNIRS than the skull. Reproducibility in terms of accuracy and precision has been desired to enable widespread clinical use of the fNIRS technique. Non-invasive continuous-wave fNIRS has not been able to achieve consistent inter-subject and intra-subject results due to its relatively low SNR and the variability of the fNIRS signal within subjects over time. Existing implementations of fNIRS, such as continuous-wave diffuse optical tomography (CW-DOT), time-domain diffuse optical tomography (TD-DOT), and broad-band fNIRS, have used fNIRS optical fiber sensors and emitters on a cap to cover the scalp, where fNIRS signals are dependent on a shape of the head. In the standard model of NSIR transmission, the light travels in an arc whose depth is dependent on the separation of the emitting and detecting probes. Short emitter-detector pairs measure extracerebral tissues, whereas long spaced emitter-detector pairs measure deeper cortical structures. It may be possible to use a signal from short emitter-detector pairs as a reference signal to subtract from a signal of long emitter detector pairs, thereby removing common-mode signals such as movement artifacts; however, such approach is ineffective to lead to a viable commercial product in the clinical domain.

### DESCRIPTION OF THE DRAWINGS

FIG. 1 is a schematic illustration of a subcutaneous fNIRS system according to some examples.

FIG. 2A is a schematic diagram of a subcutaneous fNIRS system according to some examples.

FIG. 2B is a schematic diagram of a subcutaneous fNIRS device according to examples described herein.

FIG. 3A is a schematic diagram of a power supply of a subcutaneous fNIRS device according to examples described herein.

FIG. 3B is a schematic diagram of a power supply of a subcutaneous fNIRS device according to examples described herein.

FIG. 4 is a cross-section of a subcutaneous device according to examples described herein.

FIG. 5 is a schematic illustration of impedance of liquid crystal polymer (LCP) bonded to thermoplastic polyurethane (TPU) under soak test that may be used in examples of devices described herein.

FIG. 6 is a photograph showing an example of adhesion test results using 10×10 grid pattern on a borosilicate glass (BSG) substrate that may be used in examples of devices described herein.

FIG. 7 is a schematic illustration of electrical impedance spectroscopy (EIS) of parylene C film that may be used in examples of devices described herein.

FIG. 8 is a schematic illustration of leakage current of four test structures encapsulated with parylene C that may be used in examples of devices described herein.

FIG. 9 is a schematic illustration of impedance and charge storage capacity of an array assembly that may be used in examples of devices described herein.

FIG. 10A is a schematic illustration showing impedance of a subcutaneous device at different time points.

FIG. 10B is a schematic illustration of cyclic voltammogram of a subcutaneous device at different time points.

FIGS. 11A and 11B show results of relative stability of electrical properties of a subcutaneous device under stimulation according to examples described herein.

FIG. 12 is a schematic illustration showing median impedances for an alumina and parylene bilayer coated electrode array over time of a soak test in phosphate buffered saline (PBS), according to examples described herein.

FIG. 13 is a table listing median impedances for a parylene coated electrode array and alumina and a parylene bilayer coated electrode array for three days of a soak test in PBS, according to examples described herein.

FIG. 14 is a schematic illustration including a relationship between transmitted wireless radio frequency (RF) signal strengths and frequencies monitored as a function of soak time in PBS, according to examples described herein.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of these particular embodiments. In other instances, well-known materials, components, processes, controller components, software, circuitry, timing diagrams, and/or anatomy have not been described or shown in detail in order to avoid unnecessarily obscuring the embodiments.

A technology for implementing an fNIRS system under skin has been developed. In some examples, encapsulation methods are developed to provide a fully-implanted fNIRS system that performs continuous measurements of brain activity through changes in blood oxygenation levels for real-time monitoring and closed-loop applications. Subcutaneous placement of the TD-DOT emitters and detectors enables a portable fNIRS system that becomes part of a body. For example, the fNIRS system under the skin in a subgaleal space, such as above a skull, may be free from light absorption and scattering by hair roots in the skin and emitter and detector coupling to the skin. As a result, the fNIRS system may provide relatively high signal-to-noise measurements. The fNIRS system may reduce ambient light and motion artifacts, thus the subcutaneous fNIRS system is suitable for continuous imaging.

The fNIRS system may include one or more subcutaneous fNIRS emitter and detectors (device). The fNIRS device may be insulated using organic or inorganic coatings to prevent moisture ingress. The fNIRS device may be formed as a substrate including a thin film with a plurality of sheets, such as LCP sheets. Each sheet may have a thickness ranging from 5 μm to 3 mm, thus the subcutaneous fNIRS device may fit under the skin. In some examples a thickness of a subcutaneous fNIRS device may fit within the subgaleal space between the skin and a skull. For example, the thickness of the subcutaneous fNIRS device, including a substrate may range from 40 μm to 6 mm. A surface of the substrate may be treated (e.g., encapsulated) with TPU, parylene C (e.g., chlorinated parylene), silicon carbide or atomic layer deposition (ALD) of alumina. For example, a thickness of a TPU layer may range from 5 μm to 3 mm. A thickness of parylene C may range from 3 μm to 50 μm. Silicone thickness may range from 50 μm to 6 mm.

Subcutaneous fNIRS systems including subcutaneous fNIRS devices may be used for a variety of clinical applications. Subcutaneous fNIRS devices in a subgaleal space may provide advantages from several perspectives as follows. Measurements using subcutaneous fNIRS systems with less light absorption by the hair, hair follicles and skin are more accurate and are susceptible to less noise than traditional wearable fNIRS devices. Subcutaneous fNIRS systems with stable device positioning allow continuous measurements with less recalibration, unlike wearable fNIRS systems which take set-up time and frequent calibrations. Subcutaneous fNIRS systems may have broad sensing coverage across the cortex; however, the subcutaneous fNIRS systems may be less invasive than electrocorticography (ECOG) subdural grids in contact with the brain. In some examples, a subcutaneous fNIRS system may be combined with a subgalcal ECOG electrode system or a subgaleal electroencephalogram (EEG) electrode system. Subcutaneous device with light emitters and detectors as well as an EEG electrode may perform combined electrical stimulation, EEG recording, and fNIRS measurements simultaneously for brain activity sensing. The EEG electrode may be used to deliver any combination of transcranial direct current stimulation ((DCS), transcranial alternating current stimulation (tACS), temporal interference (TI) electrical stimulation, intersection short pulse (ISP) stimulation, or other forms of electrical stimulation including charge steering.

In some examples, a subcutaneous fNIRS system can be combined with vagus nerve stimulation (VNS) or spinal cord stimulation for closed-loop feedback of existing therapies for tinnitus, pain, etc. In some examples, a patient with a subcutaneous fNIRS system may be exposed to auditory or visual stimulation. The subcutaneous fNIRS system may connect to either a cranial implant or a chest implant with wireless data communication capability, and be used in a variety of applications including brain computer interface (BCI), depression, stroke, traumatic brain injuries, tinnitus, learning and memory enhancement, etc.

The subcutaneous fNIRS device may also include an implanted window including a biocompatible material that is transparent to near-infrared wavelengths. The biocompatible material may be a polymer, ceramic, or bioengineered materials such as polymethyl methacrylate (PMMA), yttria-stabilized zirconia (YSZ) with optical clearing agents (OCAs), or any other material which is transparent for near-infrared light. In some examples, the implanted window may be a craniofacial implant for repairing a craniofacial defect or replacing other sections of bone in the body. The transparent window may replace at least a portion of a skull, to increase transparency. Thus, the transparent window allows the light from the device to pass through with less scattering of light, and increases a signal to noise ratio of the fNIRS signal.

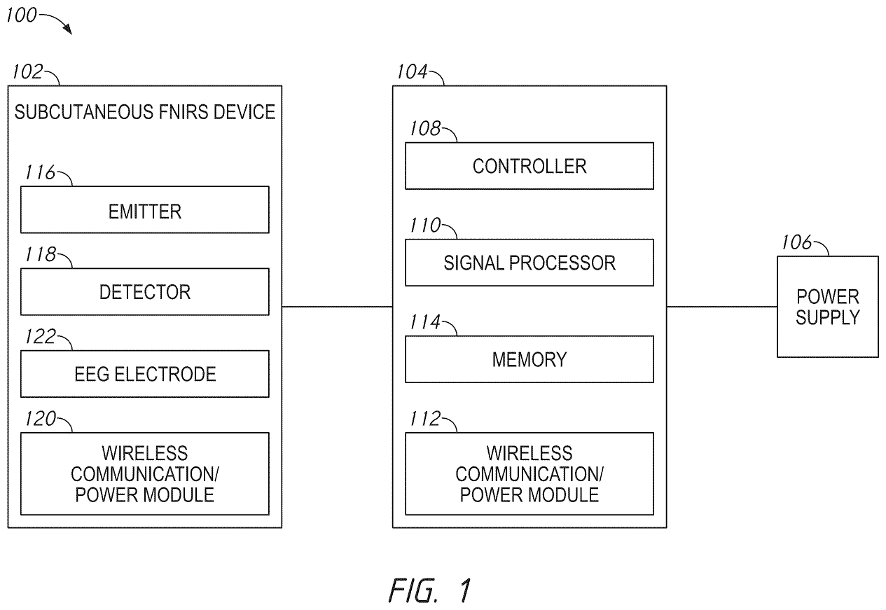

FIG. 1 is a schematic illustration of a subcutaneous fNIRS system 100 according to some examples. The subcutaneous fNIRS system 100 may include, for example, a processor and one or more memory devices, power supply, and a wireless data transmitter/receiver. In some embodiments, the subcutaneous fNIRS system 100 may include one or more subcutaneous fNIRS device 102, circuitry 104, and power supply 106. The power supply 106 may provide power for the circuitry 104. The circuitry 104 may include one or more controllers 108, one or more signal processors 110, memory 114 and a wireless communication/power module 112. In some examples, any of the processor and one or more memory devices, power supply, and the wireless data transmitter/receiver may be provided outside the circuitry 104. Each subcutaneous fNIRS device 102 may include one or more emitters 116, and one or more detectors 118. In some examples, the emitters 116 may include surface-emitting lasers. In some examples, the emitters 116 may include light emitting diodes (LEDs). In some examples, the emitters 116 may provide a plurality of wavelengths of light. For example, light having wavelengths of 680 nm, 760 nm, and 850 nm may be used for measurements of tissue oxygenation at high-resolution and high frame rates. In some examples, the detectors 118 may include photo diodes, such as single-photon avalanche diode (SPAD) arrays. In some examples, the detectors 118 may be fabricated in a semiconductor chip, such as a complementary metal-oxide-semiconductor (CMOS) chip in an integrated manner with detection electronics, such as circuits. The detectors 118 in fully integrated CMOS circuitry may provide detected fNIRS signals for high spatiotemporal resolution TD-DOT imaging. In some examples, the integrated CMOS circuitry may have a thickness that ranges from 5 nm to 50 μm. For example, resolutions of 70 picoseconds may be achieved for time-of-flight imaging using a laser pulse rate of 200 MHz with 80 mW total power. In some examples, each subcutaneous fNIRS device 102 may include a wireless communication/power module 122. In some embodiments, each of the wireless communication/power module 112 and wireless communication/power module 122 may include a low power short distance wireless communication module that may communicate signals, such as control signals and fNIRS signals, using, for example, Bluetooth, infra-red, near field communication, etc. Each of the wireless communication/power module 112 and the wireless communication/power module 122 may include a wireless transmitter/receiver. Each of the wireless communication/power module 112 and the wireless communication/power module 122 may include a wireless power charger that may receive power wirelessly. Alternatively or additionally, the subcutaneous fNIRS device 102 and the circuitry 104 may include a wired power module that may receive power in a wired manner such as a universal serial bus (USB) or some other chord, etc. The detectors 118 in the subcutaneous fNIRS device 102 may provide detected signals, such as fNIRS signals, to the signal processors 110 in the circuitry 104 via the wireless communication/power module 122 and the wireless communication/power module 112. The signal processors 110 may process the fNIRS signals and provide oxygenation and hemodynamic information for TD-DOT imaging.

The subcutaneous fNIRS system 100 may measure an oxygenation level and hemodynamic of a brain. The oxygenation level may change as areas in the brain become more active. Such brain activity may be identified in real time based on changes in blood oxygenation and other factors. The controller 108 may cause the emitters 116 to provide light. In some examples, the light may be provided as light pulses. The detectors 118 may detect the light pulses that travelled through the body part. The detectors 118 may measure time of flight of photons in the light as the light pulses propagate through and are scattered by neural tissues. By observing absorbed and/or reflected (e.g., scattered) signals at a certain wavelength of the light, absorption of hemoglobin and blood flows in certain brain regions may be measured. In some examples, the detectors 118 may include fiber optic sensors (e.g., optodes) disposed within a distance of 20-30 mm from one another. The detectors 118 may measure blood flows in between the fiber optic sensors.

In some examples, each subcutaneous fNIRS device 102 may include one or more EEG electrodes 120. In some examples, the EEG electrodes 120 may measure electrical activity of neurons underneath the EEG electrodes 120 and provide high temporal resolution, unlike fNIRS. The subcutaneous fNIRS device 102 may perform simultaneous collection (e.g., recording) of both fNIRS and EEG signals. In some examples, the EEG signals may be indicative of, for example, short-term motor imagery, whereas the fNIRS signals may be indicative of long-term changes, such as cognitive functions or pain. Thus, a user may be able to obtain short-term and long-term brain activity information from the EEG signals and fNIRS signals from the subcutaneous fNIRS device 102. In some examples, each of the EEG electrodes 120 may be smaller than each of the detectors 118, and each EEG electrode 120 may be disposed in between two adjacent detectors 118, between two adjacent emitters 116, or between two adjacent emitter 116/detector 118 pairs. Each EEG electrode 120 may detect electrical activity at a proximate spot as the detector 118 detects the fNIRS signal.

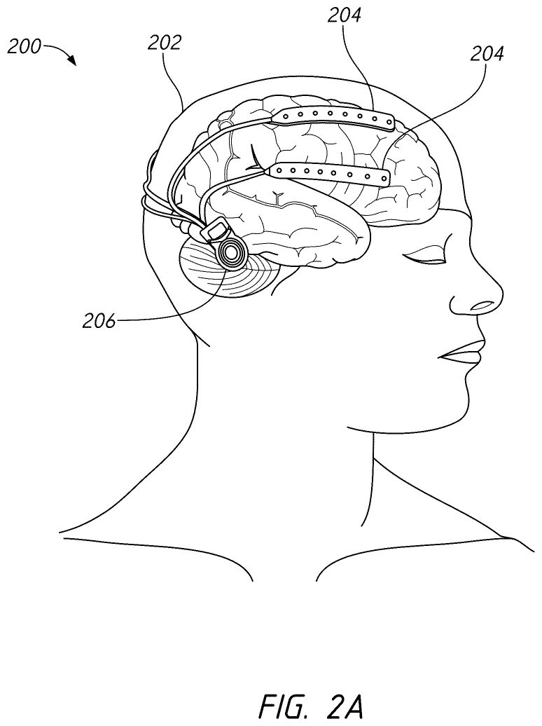

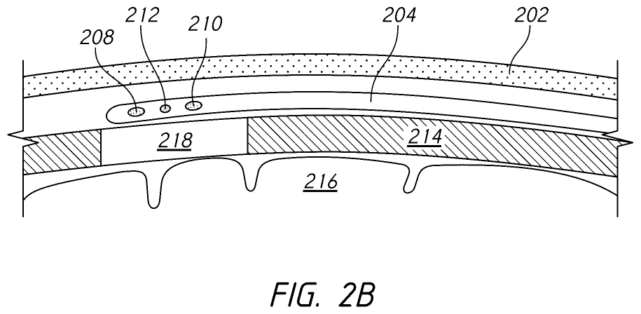

FIG. 2A is a schematic diagram of a subcutaneous fNIRS system 200 according to some examples. Generally, examples of systems described herein may include one or more subcutaneous fNIRS devices 204. In the example of FIG. 1, the subcutaneous fNIRS system 200 includes one or more subcutaneous fNIRS devices 204 and circuitry 206 coupled to the subcutaneous fNIRS devices 204. In some embodiments, the one or more subcutaneous fNIRS devices 204 may be disposed under the skin 202. In some examples, the fNIRS devices 204 may be disposed on or above the skin 202. Examples of a subcutaneous fNIRS system 200 are described in further detail in regard to FIG. 2B. While the subcutaneous fNIRS devices 204 in FIG. 2A are linear strips, the subcutaneous fNIRS devices 204 may be implemented in different shapes suitable for measurements of signals.

FIG. 2B is a schematic diagram of a subcutaneous fNIRS device 204 according to examples described herein. In some embodiments, each subcutaneous fNIRS device 204 may include one or more emitters 208 and one or more detectors 210. In some examples, each subcutaneous fNIRS device 204 may further include one or more EEG electrodes 212. In some examples, the subcutaneous fNIRS device 204 may be disposed in a subgaleal space under the skin 202 and on or above a skull 214 protecting a brain 216. In some examples, the emitters 208 and the detectors 210 may be the emitters 116 and the detectors 118 of FIG. 1. In some examples, the subcutaneous fNIRS device 204 may include tapering (not shown) on edges that reduces exposure of the subcutaneous fNIRS device 204 to outside the skin.

In some embodiments, an implanted window 218 may be disposed on the skull 214. In some examples, the subcutaneous fNIRS device 204 may be placed on the skull in a manner that some of the emitters 208, the detectors 210, and/or the EEG electrodes 212 may be placed on or above the implanted window 218. In some examples, the implanted window 218 may be a craniofacial implant for repairing a craniofacial defect or replacing other sections of bone in the body. For example, the implanted window 218 may include one or more transparent biocompatible materials, for example, transparent PMMA plastic, YSZ ceramic with OCAs, or any other transparent biocompatible material suited for safe use in craniofacial reconstruction. While a transparent PMMA craniofacial implant is the preferred embodiment, the prefabricated transparent custom craniofacial implant may include a polymer, metal, bioengineered material, or any combinations thereof for which may also be substantially transparent. In some examples,

PMMA, YZS with OCAs, and similar ceramic and polymer materials are transparent to near infrared light signals having a wavelength that ranges from 800 nm to 2500 nm. In some examples, transparent implanted window 218 may replace at least a portion of a skull, to increase transparency. Thus, the transparent window allows the light from the device to pass through with less scattering of light, and increases a signal to noise ratio of the fNIRS signal.

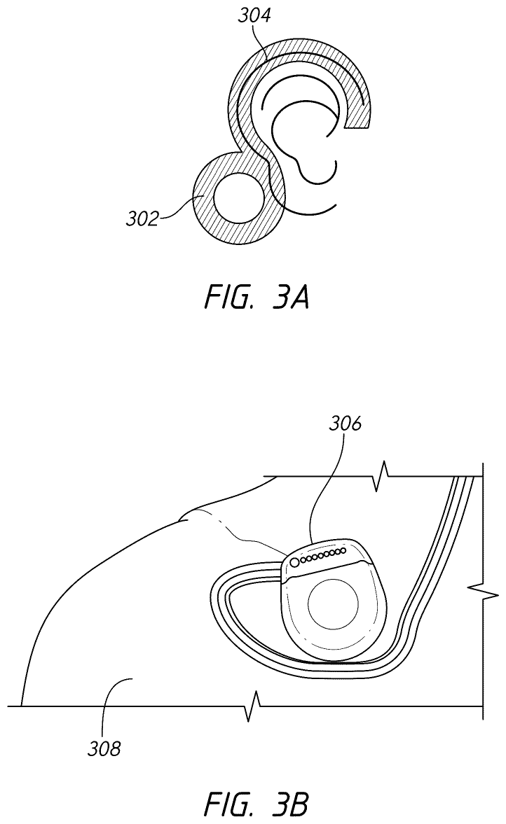

In some examples, a power supply for a subcutaneous fNIRS system may be outside the circuitry 206. In some examples, a power supply may be disposed on an car. FIG. 3A is a schematic diagram of a power supply 302 of a subcutaneous fNIRS system 200 according to examples described herein. The power supply 302 may be disposed on or embedded under the skin of an car 304. The power supply 302 may supply power to the subcutaneous fNIRS device 204 via the circuitry 206.

In some examples, a power supply may be disposed on a chest. FIG. 3B is a schematic diagram of a power supply 306 of a subcutaneous fNIRS system 200 according to examples described herein. The power supply 306 may be disposed on or embedded under the skin of a chest 308. The power supply 306 may supply power to the subcutaneous fNIRS device 204 via the circuitry 206.

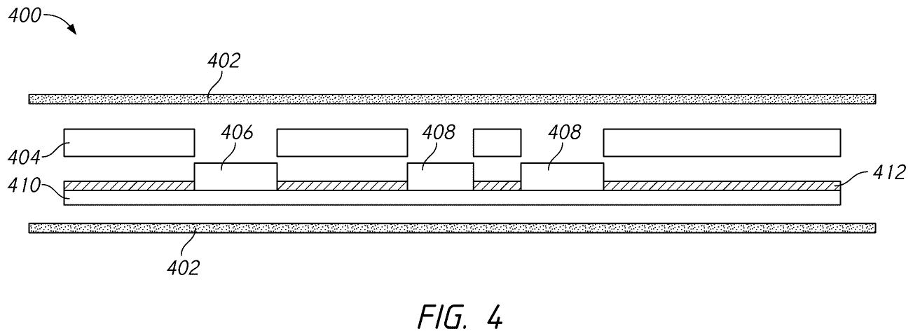

FIG. 4 is a cross-section of a subcutaneous device 400 according to examples described herein. In FIG. 4, the subcutaneous device 400 may be implemented as a semiconductor chip. The subcutaneous device 400 may include top and bottom heating bars 402, stencils 404, one or more emitters 406 and one or more detectors 408 on a substrate 410. A face of the substrate 410 not covered with the emitters 406 and the detectors 408 may be covered with a surface 412 by surface treatment. In some examples, the emitters 406 and the detectors 408, such as the emitters 116 and the detectors 118, may be encapsulated in the substrate 410 including polymer.

The emitters 406 may include surface-emitting lasers or LEDs. The detectors 408 may include photodiodes, such as single-photon avalanche diode (SPAD) arrays. In some embodiments, metal such as gold, silver or copper may be used. In some examples, to improve stability in vivo chronically, metal such as platinum, iridium, iridium oxide, or any combination thereof may be used as conducting material for device pads (e.g., microcontacts) on the substrate 410. Additionally, gold, iridium oxide and/or platinum-iridium may be used for device pads if ECOG sensors are further included in the substrate 410.

The stencils 404 may be designed to prevent direct stress onto the components. The stencils 404 may cover the substrate 410, except the emitters 406 and the detectors 408, to provide light paths. The stencils 404 may be processed with micro-edging around the emitters 406 and the detectors 408. The micro-edging in conjunction with precise alignment during a manufacturing process of the subcutaneous device 400 may reduce an amount of stress to be applied to the components.

In some examples, the substrate 410 may be a polyimide substrate. In some examples, the substrate 410 may be a flexible liquid crystal polymer thin-film (LCP-TF) substrate including LCP. In some examples, the LCP-TF substrate may have a thickness that may range from less than 10 μm to 2.5 mm. A substrate including LCP may have longevity and low water permeability compared to a polyimide substrate (e.g., up to 25 times less than polyimide substrates) and reliability and lifetime of an implanted array in the substrate may be extended. In some examples, the substrate may include TPU. TPU has been used in the medical industry due to several properties. For example, TPU has water, fungus, and abrasion resistance. TPU's rubber-like elasticity ensures flame retardancy at varying opacities. TPU polymers having robust mechanical properties, durability, chemical and oil resistance, and biocompatibility are highly desirable for implantable devices. TPU polymers having customizable mechanical properties (e.g., hardness from 72 A to 60 D Shore durometers) and chemistries (e.g., hydrophilic and hydrophobic) may provide precise control over drug elution and compatibility may be possible. For example, a thickness of a TPU layer may range from 5 μm to 3 mm. In some examples, the substrate 410 may include silicone. For example, a thickness of silicone may range from 50 μm to 6 mm. In some examples, the substrate 410 may include any combination of LCP, silicone or TPU thereof.

In some examples, the subcutaneous device 400 may be an LCP-TF device. An LCP-TF device has demonstrated great stability during accelerated aging and in vivo implantation tests. In some examples, an LCP-TF device including two LCP sheets, each having less than 30 μm (e.g., 25 μm) thickness, with wirings in between, may be fused into a single layer at 282° C. under 100 pounds per square inch of force. The LCP-TF device may have resistance to delamination, compared to polyimide device arrays suffering from delamination due to their reliance on inter-layer adhesion. A soak test at 60° C. revealed a lifetime exceeding five years at 37° C. equivalence. In some examples employing rats and non-human primates, implanted LCP-TF device demonstrated chronic signal reliability and stable performance for a longer lifetime than polyimide device.

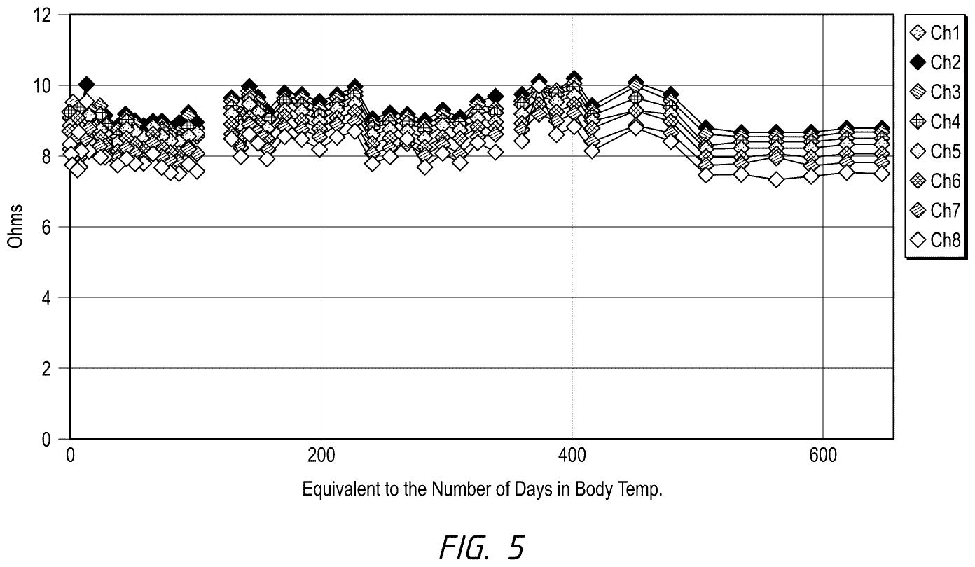

Furthermore, LCP bonding to TPU may provide material properties of both TPU and LCP, ensuring robustness even during accelerated aging conditions. The bonding procedure may include applying heat and pressure to the substrate 410 to melt and blend the two materials together. In some examples, a hydraulic press equipped with heating plates may be used to bond a sandwich structure together. Thorough cleaning, pre-processing, and precise alignment of the silicone, teflon, TPU, LCP device and stainless sheet (SS) sheet may ensure correct bonding profiles and seamless mold release of the LCP and TPU. Once the pressure is released, the entire setup is removed from the press, and a mold is disassembled to retrieve the bonded profiles. The bonded profiles may be cut using a laser marker, followed by removal of excess TPU. Subsequently, the subcutaneous device 400 may be fabricated. A soak test of the LCP bonded to TPU was performed under 37° C. FIG. 5 is a schematic illustration of impedance of the LCP bonded to TPU under a soak test that may be used in examples of devices described herein. In some examples, a subcutaneous device may include a plurality of electrodes. In FIG. 5, an example device including eight electrodes providing eight channels was tested at 37° C., which is normal body temperature. Therefore, the aging of the device was at a normal speed (i.e. 1 day of testing equaled 1 day in the body at room temperature). Impedance values of the eight electrodes (Ch1-Ch8) were relatively stable (around 8Ω) over 400 days. The LCP bonded to TPU demonstrated great impedance stability.

As shown above, the emitters 406 and the detectors 408 may be implemented in a size to provide TD-DOT for subcutaneous (e.g., subgaleal) implantation. By disposing the subcutaneous device 400 between two polyurethane sheets to be blended and melted together to protect a chip, such as the subcutaneous device 400 from fluid ingress. LCP can also be used in conjunction with TPU. The fusing process can be performed using a heated hydraulic press as detailed in the preliminary data shared on fusing LCP and TPU constructs. The TPU encapsulated chip can then be laser cut to the appropriate dimensions.

Surface treatment may be performed on the substrate 410 and the surface 412 may be formed on the substrate 410. The surface 412, including at least one of encapsulation and stencils 404 covering components including the emitters 406 and the detectors 408, may protect the components from any pressing and heating applied to the substrate 410. For example, encapsulation around each component may provide a cushion that may reduce stress onto the component. Encapsulation may use at least one of TPU, parylene C, silicon carbide, or ALD of alumina that excel in water permeability, mechanical strength, and overall stability. In some examples, the surface treatment may provide an ionic barrier between physiological fluids and components to be insulated. For example, a thickness of a TPU layer may range from 5 μm to 3 mm. A thickness of parylene C may range from 3 μm to 50 μm. Silicone thickness may range from 50 μm to 6 mm.

Robust parylene C encapsulation methods may result in suitable adhesion and electrical insulation for encapsulation of the subcutaneous device 400. Parylene C having chemical inertness, low dielectric constant (εᵣ=3.15), high resistivity (˜10¹⁵ Ω·cm), and a relatively low water vapor transmission rate of 0.2 g·mm/m²·day, has been a reliable coating material for biomedical implantable devices. In some examples, parylene C may be applied by chemical vapor deposition (CVD) at room temperature to form a conformal, pin-hole-free film, without solvents. Moreover, parylene C has ion barrier properties for neural interfaces exposed to physiological fluids.

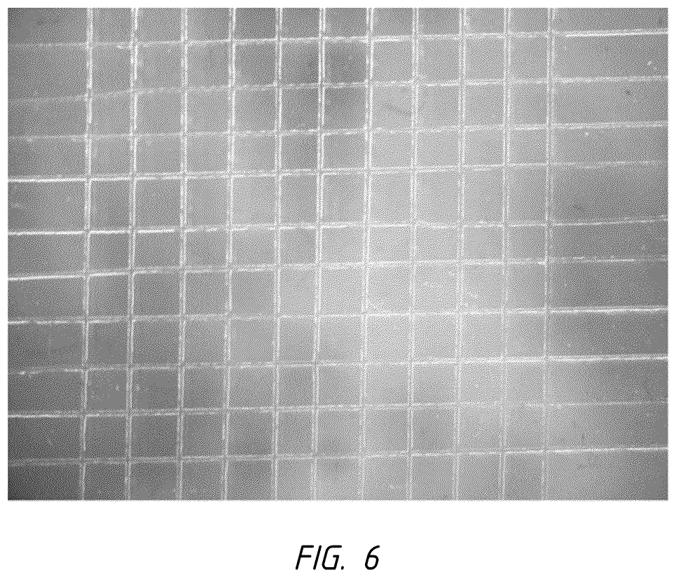

By using an adhesion promoter, such as methacryloxy functional trimethoxy silane (e.g., Silquest A-174 silane), may enhance adhesion of parylene C film. This enhanced adhesion may be observed with both silicon and BSG substrates achieving grade 5B adhesion in a tape adhesion test, whereas substrates without the adhesion promoter exhibited a grade of OB. FIG. 6 is a photograph showing an example of adhesion test results using a 10×10 grid pattern on a BSG substrate that may be used in examples of devices described herein. All the 3 to 4.5 μm thick parylene C squares made by the cuts remained on the BSG substrate. The pitch of the cuts is 1 mm. Assessment of adhesion results under the ASTM D3359 B standard highlights improvements in adhesion of 3 to 4.5 μm thick parylene C layers when using the adhesion promoter. The adhesion promoter effectively mitigates delamination of the encapsulation and ensures bonding.

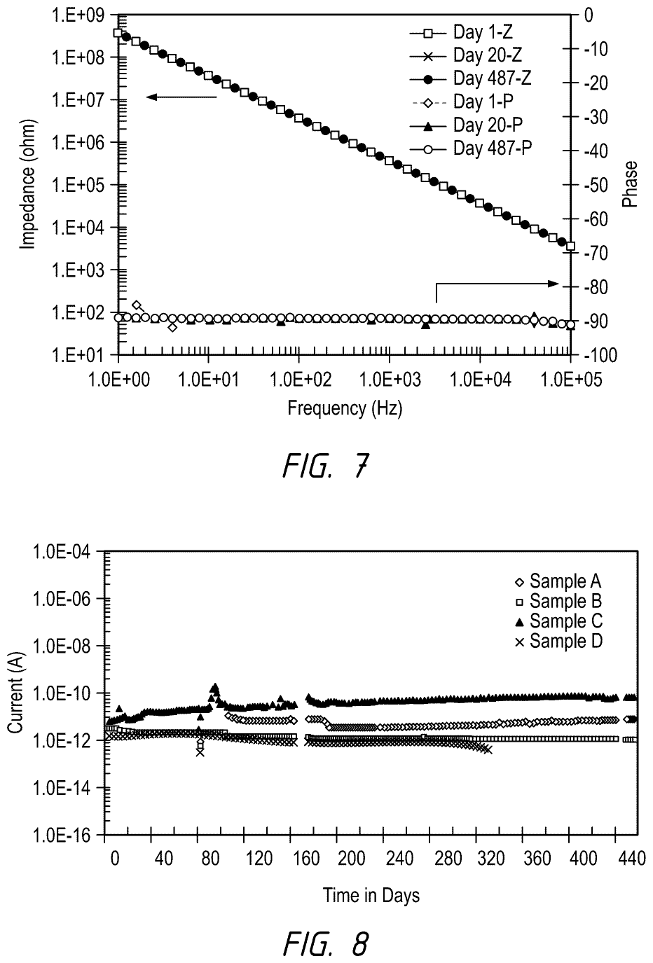

Electrical performance of parylene C layers was tested using electrical impedance spectroscopy (EIS) and leakage current tests. FIG. 7 is a schematic illustration of EIS of parylene C film that may be used in examples of devices described herein. The parylene C layers may be selected over a 487-day soaking test in 37° C. saline. The impedance was denoted by Z and the phase was denoted by P in FIG. 7. The EIS observed a phase angle of −90° that implies a capacitor behavior, indicating absence of cracks or pinholes in the encapsulation film. The consistency in capacitance across different time points strongly suggests absence of degradation or water absorption within the film.

FIG. 8 is a schematic illustration of leakage current of four test structures encapsulated with parylene C that may be used in examples of devices described herein. In this example, a thickness of parylene C is less than 4.5 μm. Test samples were applied with a 5 V_{dc} bias, and the leakage currents were monitored as a function of time for more than one year. Sample D was removed after 320 days due to failure of an electrical connection. Upon immersion in saline, the leakage currents displayed a slight increase but consistently remained below 10\hat{ }⁻¹⁰ A throughout the 450-day testing period. This leakage behavior indicates that the presence of the 5 V_{dc} bias on samples immersed in saline did not compromise the integrity of the parylene C encapsulation.

The EIS and leakage current test results of FIGS. 7 and 8 show that parylene C is a good electrical insulator for neural interface devices and that parylene C may provide insulation at 37° C. saline under biased or nonbiased conditions for more than a year.

In some examples, the surface treatment, including, for example, ALD of alumina or silicon carbide, may be used to improve electrical properties and stability of the subcutaneous device 400 of FIG. 4.

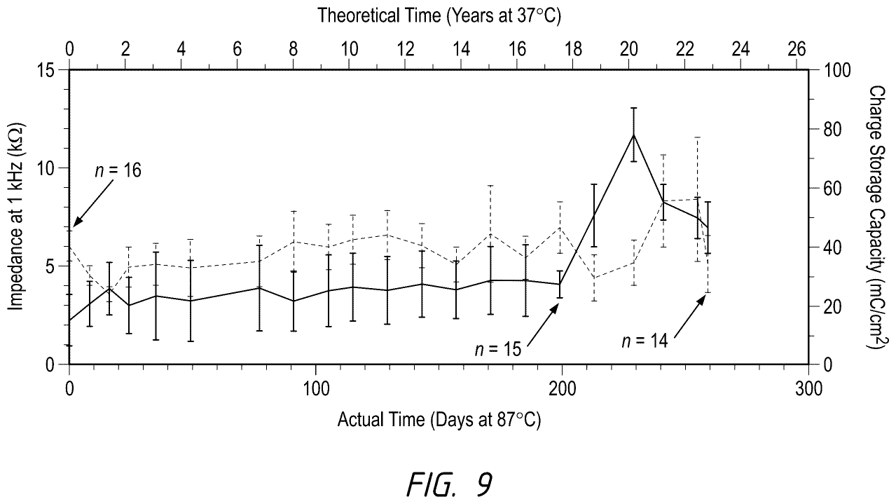

In some examples, silicon carbide may be used to enhance the electrical properties and stability at tissue interfaces of subgaleal devices. A high-channel count microelectrode array, such as the Utah electrode array (UEA), may be coated with silicon carbide, and its electrical properties and stability may be tested over time. The array assembly was soaked in phosphate buffered saline (PBS) at 87° C., and the array impedance at 1 kHz and the array's charge storage capacity (CSC) were measured. Numbers of samples are 15 and 14, respectively. FIG. 9 is a schematic illustration of the impedance and CSC of the array assembly that may be used in examples of devices described herein. FIG. 9 shows stability of electrical properties of the array coated with silicon carbide over 250 days at 87° C., theoretically equivalent to 22 years at 37° C.

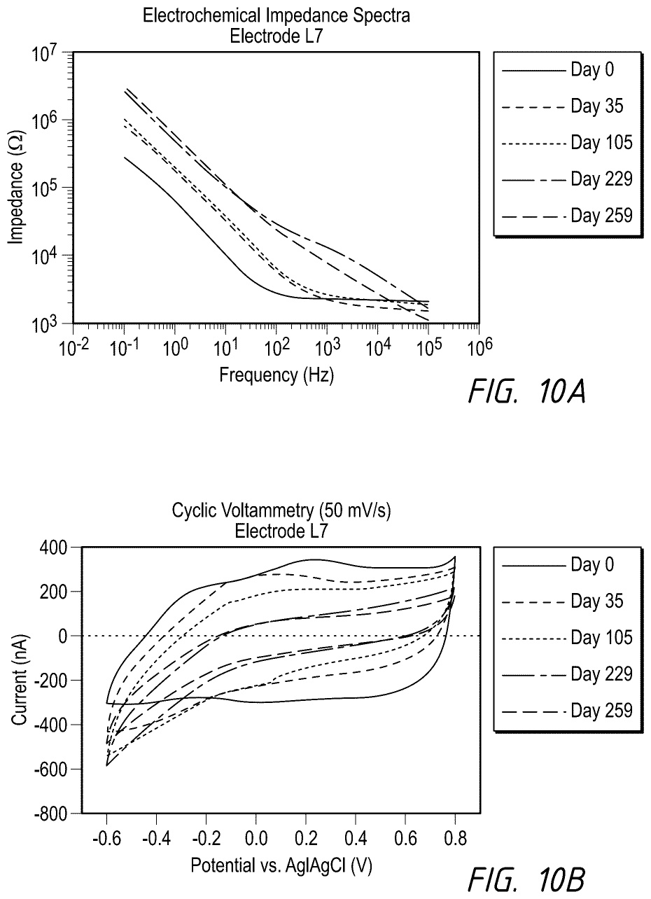

Cyclic voltammetry and impedance at different time points of a subcutaneous device going through aging are obtained. FIG. 10A is a schematic illustration of showing the impedance of a subcutaneous device at different time points. FIG. 10B is a schematic illustration of a cyclic voltammogram of a subcutaneous device at different time points.

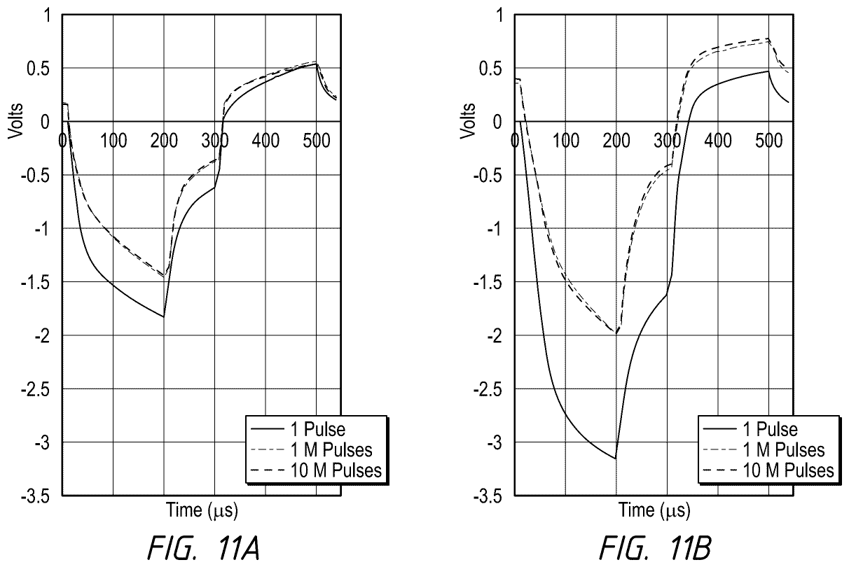

The stability of the silicon carbide coating during stimulation was investigated since it may sometimes drive unwanted chemical and physical reactions which damage the devices. FIGS. 11A and 11B show results of relative stability of electrical properties of a subcutaneous device under stimulation according to examples described herein. Electrode sites on two different UEAs (A and B) were subjected to 10 million stimulation pulses and voltage transients were recorded. The amplitude of the voltage transient decreased within the first 1 million pulses, then remained stable for 10 million pulses.

Mechanical stability of the silicon carbide was investigated through an electrode insertion test and a wire bend test. The insertion of a UEA into 1% agarose-PBS gel (model cortex material) resulted in no damage to devices tested. The devices that were damaged prior to insertion retained their structure and no additional damage was observed.

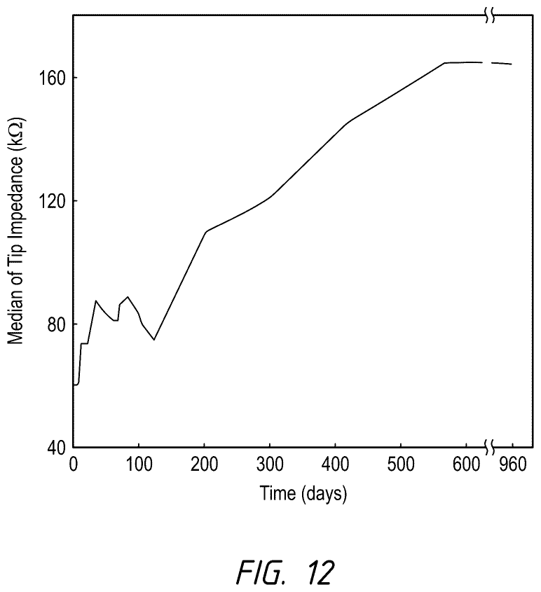

In some examples, ALD of alumina may be used to enhance the electrical properties and stability at the tissue interface in subgaleal devices. For example, ALD of alumina may also be deposited prior to parylene C encapsulation in order to improve the impedance, signal stability and strength, and current draw long term reliability. FIG. 12 is a schematic illustration showing median impedances for an alumina and parylene bilayer coated electrode array over time of a soak test in PBS at 37° C., according to examples described herein. (“Long-term reliability of Al2O3 and Parylene C bilayer encapsulated Utah electrode array based neural interfaces for chronic implantation”, J. Neural Eng. 11 (2014)). Different from the trend of continuous drop in impedance for parylene coated arrays, median impedances of alumina and -parylene bilayer coated wired arrays increased from 60 kΩ to 160 kΩ after 960 equivalent days of soak testing at 37° C. For bilayer coated arrays, the loss of iridium oxide and etching of silicon in PBS solution (leading to impedance increase) dominates over the slow bilayer encapsulation degradation (resulting in decreased impedance).

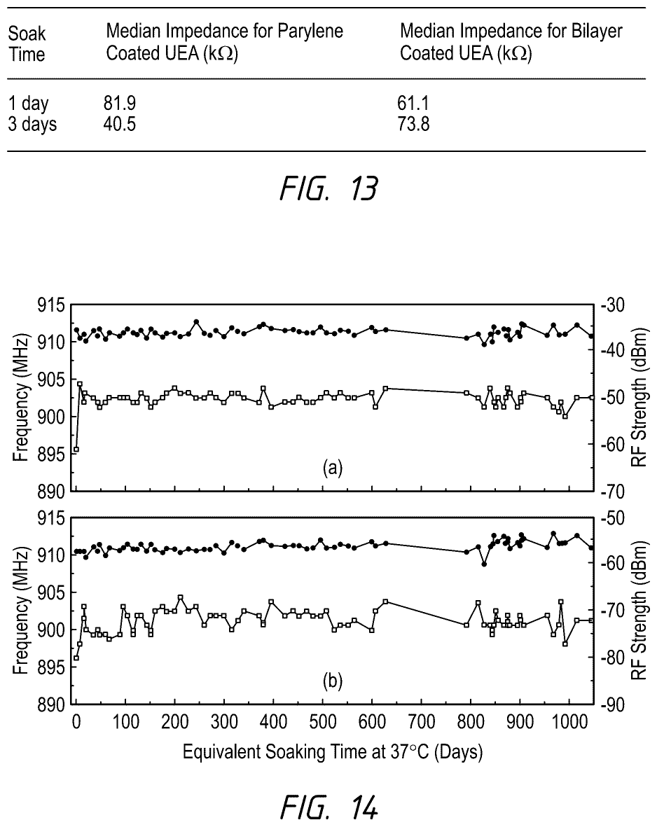

In an accelerated lifetime test, wired, wireless UEAs and active arrays were soaked in PBS at 57° C. FIG. 13 is a table listing median impedances for parylene coated UEA and alumina and parylene bilayer coated UEA for three days of a soak test in PBS, according to examples described herein. Id.

FIG. 14 is a schematic illustration including a relationship between transmitted wireless RF signal strengths and frequencies monitored as a function of soak time in PBS according to examples described herein. In (a) of FIG. 14, signals are extracted from customized wireless unit, and in (b) of FIG. 14, signals were measured using a spectrum analyzer. In both measurement methods, the RF signal strengths and corresponding frequencies stayed relatively stable during 1044 days of equivalent soak time at 37° C. Bilayer coated wireless UEAs incorporated with active electronics had stable power-up frequencies of ˜910 MHz and constant RF signal strengths of ˜−50 dBm (measured by hand receiver) over 1044 equivalent days of soak testing at 37° C., showing the slow water ingress and excellent insulation performance of the bilayer encapsulation. The current draw of active arrays was constant at ˜3 mA with a power supply of V_{dd} at 1.5V and Vₛₛ at −1.5V during 228 equivalent days of soak testing at 37° C. The low and constant current draw is a reliable indication of good protection of the device by the encapsulation. Based on the coating performance on neural interfaces, the bilayer encapsulation may be used for many other chronic biomedical implantable devices to increase lifetime of the devices.

The subcutaneous fNIRS techniques and methods to manufacture subcutaneous fNIRS devices described herein may be used for treatment confirmation and may further be extended to chronic monitoring of brain activities.

Examples provided herein of both the design of subcutaneous fNIRS systems including subcutaneous fNIRS devices and the clinical applications are not the limit of the uses of the subcutaneous fNIRS technique in the subgaleal zones. Many configurations of subcutaneous fNIRS systems exist, as well as applications that would benefit from the use of the technology described herein.

It is to be appreciated that any one of the above embodiments or processes may be combined with one or more other embodiments and/or processes or be separated and/or performed amongst separate devices or device portions in accordance with the present systems, devices and methods.

Finally, the above discussion is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

What is claimed is:

1. An apparatus comprising:
a substrate and a device configured to be disposed under skin, the device comprising:
one or more emitters disposed on or in the substrate and configured to emit light, the light being configured to propagate a body part under skin; and
one or more light detectors disposed on or in the substrate and configured to detect changes in light absorption and scattering through the body part and further configured to provide fNIRS signals.

2. The apparatus of claim 1, wherein a thickness of the apparatus ranges from 40 μm to 6 mm.

3. The apparatus of claim 1, further comprising tapering on edges of the device, the tapering configured to reduce the visibility of the device contour from outside the skin.

4. The apparatus of claim 1, further comprising a surface on the substrate, wherein the surface includes at least one of TPU, parylene C, silicon carbide or ALD of alumina, epoxy, or other organic or inorganic coatings to prevent moisture ingress, or any combination thereof.

5. The apparatus of claim 1, wherein the substrate comprises at least one of the LCP, silicone or TPU layers or any combination thereof.

6. The apparatus of claim 5, further comprising an LCP layer comprising a plurality of LCP sheets.

7. The apparatus of claim 6, wherein each LCP sheet of the plurality of LCP sheets has a thickness ranging between 5 μm to 3 mm.

8. The apparatus of claim 1, wherein the device is sandwiched between two or more LCP sheets.

9. A method comprising:
forming a substrate;
performing surface treatment on the substrate, the surface treatment including encapsulating a surface of the substrate; and
forming one or more emitters and one or more light detectors on the substrate.

10. The method of claim 9, wherein forming the substrate includes bonding LCP to TPU.

11. The method of claim 9, wherein performing the surface treatment includes encapsulating the surface with at least one of TPU, parylene C, silicon carbide or ALD of alumina.

12. The method of claim 11, wherein encapsulating the surface with parylene C comprises using an adhesion promoter.

13. A system comprising:
a subcutaneous device configured to be disposed under skin, the subcutaneous device including:
one or more emitters configured to emit light, the light being configured to propagate a body part under the skin; and
one or more light detectors configured to detect changes in light absorption and scattering through the body part and further configured to provide fNIRS signals, and
a processor configured to receive the detected changes from the one or more light detectors, to process the detect changes to obtain at least one of oxygenation and hemodynamic information responsive to the detected changes, and further configured to monitor the at least one of oxygenation and hemodynamic information.

14. The system of claim 13, wherein the subcutaneous device further comprises one or more EEG electrodes configured to obtain an EEG signal.

15. The system of claim 14, wherein each EEG electrode of the one or more EEG electrodes is disposed between adjacent light detectors of the one or more light detectors.

16. The system of claim 13, further comprising a window including at least one transparent material,
wherein the subcutaneous device is disposed on the window, and
wherein the window having a higher transparency than the body part is configured to allow transmission of the light from the one or more emitters to a target portion of the body part.

17. The system of claim 16, wherein the transparent material comprises polymethyl methacrylate (PMMA).

18. The system of claim 13, wherein the subcutaneous device is a subgaleal device configured to be disposed on or above a skull of a head, and
wherein the light is configured to propagate through the skull and a brain of the head.

19. The system of claim 18, further comprising a window replacing at least a portion of the skull, the window having a higher transparency than the skull, configured to allow the light from the subcutaneous device to pass through with less scattering of light than the skull.
